(19) United States
(12) Patent Application Publication
(10) Pub. No.: US 2009/0215345 A1
(43) Pub. Date: Aug. 27, 2009

(54) PARTICLE-CONTAINING FIBROUS WEB

(75) Inventors: Larry A. Brey, Woodbury, MN (US); Andrew S. Viner, Roseville, MN (US); Marvin E. Jones, Grant, MN (US); John E. Trend, St. Paul, MN (US); Raymond Senkus, Hudson, WI (US); Mary E. Senkus, Hudson, WI (US); Thomas I. Insley, Lake Elmo, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 12/436,358

(22) Filed: May 6, 2009

Related U.S. Application Data
(62) Division of application No. 10/983,770, filed on Nov. 8, 2004.

(51) Int. Cl. B01J 20/28; D04H 13/00; A61L 15/42

(52) U.S. Cl. 442/417

(57) ABSTRACT

A porous sheet article comprising a self-supporting nonwoven web of polymeric fibers and at least 80 weight percent sorbent particles enmeshed in the web, the fibers having sufficiently greater elasticity or sufficiently greater crystallization shrinkage than similar caliper polypropylene fibers and the sorbent particles being sufficiently evenly distributed in the web so that the web has an Adsorption Factor A of at least 1.6×10⁴/mm water. The articles have low pressure drop and can provide filter elements having long service life and an Adsorption Factor approaching and in some instances exceeding that of a packed carbon bed.

This application is a divisional of U.S. Ser. No. 10/983,770, filed Nov. 8, 2004, the disclosure of which is incorporated by reference in its entirety herein.

This invention relates to particle-containing fibrous webs and filtration.

### BACKGROUND

Respiratory devices for use in the presence of solvents and other hazardous airborne substances sometimes employ a filtration element containing sorbent particles. The filtration element may be a cartridge containing a bed of the sorbent particles or a layer or insert of filtration material impregnated or coated with the sorbent particles. Design of the filtration element may involve a balance of sometimes competing factors such as pressure drop, surge resistance, overall service life, weight, thickness, overall size, resistance to potentially damaging forces such as vibration or abrasion, and sample-to-sample variability. Packed beds of sorbent particles typically provide the longest service life in the smallest overall volume, but may exhibit higher than optimal pressure drop. Fibrous webs loaded with sorbent particles often have low pressure drop but may also have low service life, excessive bulk or larger than desirable sample-to-sample variability.

References relating to particle-containing fibrous webs include U.S. Pat. Nos. 2,988,469 (Watson), 3,971,373 (Braun), 4,429,001 (Kolpin et al.), 4,681,801 (Eian et al.), 4,741,949 (Morman et al.), 4,797,318 (Brooker et al. '318), 4,948,639 (Brooker et al. '639), 5,035,240 (Braun et al. '240), 5,328,758 (Markell et al.), 5,720,832 (Minto et al.), 5,972,427 (Mühlfeld et al.), 5,885,696 (Groeger), 5,952,092 (Groeger et al. '092), 5,972,808 (Groeger et al. '808), 6,024,782 (Freund et al.), 6,024,813 (Groeger et al. '813), 6,102,039 (Springett et al.) and PCT Published Application Nos. WO 00/39379 and WO 00/39380. References relating to other particle-containing filter structures include U.S. Pat. Nos. 5,033,465 (Braun et al. '465), 5,147,722 (Koslow), 5,332,426 (Tang et al.) and 6,391,429 (Senkus et al.). Other references relating to fibrous webs include U.S. Pat. No. 4,657,802 (Morman).

### SUMMARY OF THE INVENTION

Although meltblown nonwoven webs containing activated carbon particles can be used to remove gases and vapors from air, it can be difficult to use such webs in replaceable filter cartridges for gas and vapor respirators. For example, when webs are formed from meltblown polypropylene and activated carbon particles, the readily-attainable carbon loading level ordinarily is about 100 to 200 g/m². If such webs are cut to an appropriate shape and inserted into replaceable cartridge housings, the cartridges may not contain enough activated carbon to meet capacity requirements set by the applicable standards-making bodies. Although higher carbon loading levels may be attempted, the carbon particles may fall out of the web thus making it difficult to handle the web in a production environment and difficult reliably to attain a targeted final capacity. Post-formation operations such as vacuum forming can also be employed to densify the web, but this requires additional production equipment and extra web handling.

We have found that by fabricating a highly-loaded particle-containing nonwoven web using a suitably elastic or suitably shrink-prone polymer, we can obtain a porous sheet article having a very desirable combination of high service life and low pressure drop. The resultant webs can have relatively low carbon shedding tendencies and can be especially useful for mass producing replaceable filter cartridges using automated equipment.

The present invention provides, in one aspect, a porous sheet article comprising a self-supporting nonwoven web of polymeric fibers and at least 80 weight percent sorbent particles enmeshed in the web, the fibers having sufficiently greater elasticity or sufficiently greater crystallization shrinkage than similar caliper polypropylene fibers and the sorbent particles being sufficiently evenly distributed in the web so that the web has an Adsorption Factor A of at least 1.6×10⁴/mm water (viz., at least 1.6×10⁴ mm water⁻¹).

In another aspect, the invention provides a process for making a porous sheet article comprising a self-supporting nonwoven web of polymeric fibers and sorbent particles, comprising:

a) flowing molten polymer through a plurality of orifices to form filaments;

b) attenuating the filaments into fibers;

c) directing a stream of sorbent particles amidst the filaments or fibers; and

d) collecting the fibers and sorbent particles as a nonwoven web

wherein at least 80 weight percent sorbent particles are enmeshed in the web and the fibers have sufficiently greater elasticity or sufficiently greater crystallization shrinkage than similar caliper polypropylene fibers and the sorbent particles being sufficiently evenly distributed in the web so that the web has an Adsorption Factor A of at least 1.6×10⁴/l mm water.

In another aspect the invention provides a respiratory device having an interior portion that generally encloses at least the nose and mouth of a wearer, an air intake path for supplying ambient air to the interior portion, and a porous sheet article disposed across the air intake path to filter such supplied air, the porous sheet article comprising a self-supporting nonwoven web of polymeric fibers and at least 80 weight percent sorbent particles enmeshed in the web, the fibers having sufficiently greater elasticity or sufficiently greater crystallization shrinkage than similar caliper polypropylene fibers and the sorbent particles being sufficiently evenly distributed in the web so that the article has an Adsorption Factor A of at least 1.6×10⁴/mm water.

In yet another aspect the invention provides a replaceable filter element for a respiratory device, the element comprising a support structure for mounting the element on the device, a housing and a porous sheet article disposed in the housing so that the element can filter air passing into the device, the article comprising a self-supporting nonwoven web of polymeric fibers and at least 80 weight percent sorbent particles enmeshed in the web, the fibers having sufficiently greater elasticity or sufficiently greater crystallization shrinkage than similar caliper polypropylene fibers and the sorbent particles being sufficiently evenly distributed in the web so that the element has an Adsorption Factor A of at least 1.6×10⁴/mm water.

These and other aspects of the invention will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWING

FIG. 1 is a schematic cross-sectional view of a disclosed porous sheet article;

FIG. 2 is a schematic cross-sectional view of a disclosed multilayer porous sheet article;

FIG. 3 is a schematic view, partially in cross-section, of a disclosed replaceable filter element;

FIG. 4 is a perspective view of a disclosed respiratory device utilizing the element of FIG. 3;

FIG. 5 is a perspective view, partially cut away, of a disclosed disposable respiratory device utilizing the porous sheet article of FIG. 1;

FIG. 6 is a schematic cross-sectional view of a meltblowing apparatus for making porous sheet articles.

FIG. 7 is a schematic cross-sectional view of a spun bond process apparatus for making porous sheet articles.

FIG. 8 is a schematic cross-sectional view of another meltblowing apparatus for making porous sheet articles.

FIG. 9 and FIG. 10 are graphs showing service life comparisons.

Like reference symbols in the various figures of the drawing indicate like elements. The elements in the drawing are not to scale.

### DETAILED DESCRIPTION

As used in this specification with respect to a sheet article, the word “porous” refers to an article that is sufficiently permeable to gases so as to be useable in a filter element of a personal respiratory device.

The phrase “nonwoven web” refers to a fibrous web characterized by entanglement or point bonding of the fibers.

The term “self-supporting” refers to a web having sufficient coherency and strength so as to be drapable and handleable without substantial tearing or rupture.

The phrase “attenuating the filaments into fibers” refers to the conversion of a segment of a filament into a segment of greater length and smaller diameter.

The word “meltblowing” means a method for forming a nonwoven web by extruding a fiber-forming material through a plurality of orifices to form filaments while contacting the filaments with air or other attenuating fluid to attenuate the filaments into fibers and thereafter collecting a layer of the attenuated fibers.

The phrase “melt blown fibers” refers to fibers made using meltblowing. The aspect ratio (ratio of length to diameter) of melt blown fibers is essentially infinite (e.g., generally at least about 10,000 or more), though melt blown fibers have been reported to be discontinuous. The fibers are long and entangled sufficiently that it is usually not possible to remove one complete melt blown fiber from a mass of such fibers or to trace one melt blown fiber from beginning to end.

The phrase “spun bond process” means a method for forming a nonwoven web by extruding a low viscosity melt through a plurality of orifices to form filaments, quenching the filaments with air or other fluid to solidify at least the surfaces of the filaments, contacting the at least partially solidified filaments with air or other fluid to attenuate the filaments into fibers and collecting and optionally calendaring a layer of the attenuated fibers.

The phrase “spun bond fibers” refers to fibers made using a spun bond process. Such fibers are generally continuous and are entangled or point bonded sufficiently that it is usually not possible to remove one complete spun bond fiber from a mass of such fibers.

The phrase “nonwoven die” refers to a die for use in meltblowing or the spun bond process.

The word “enmeshed” when used with respect to particles in a nonwoven web refers to particles that are sufficiently bonded to or entrapped within the web so as to remain within or on the web when the web is subjected to gentle handling such as draping the web over a horizontal rod.

The phrase “elastic limit” when used with respect to a polymer refers to the maximum distortion that a body formed from the polymer can undergo and return to its original form when relieved from stress.

The words “elastic” or “elasticity” when used with respect to a polymer refer to a material that has an elongation at its elastic limit of greater than about 10% as measured using ASTM D638-03, Standard Test Method for Tensile Properties of Plastics.

The phrase “crystallization shrinkage” refers to the irreversible change in length of an unconstrained fiber that may occur when the fiber passes from a less ordered, less crystalline state to a more ordered, more crystalline state, e.g. due to polymer chain folding or polymer chain rearrangement.

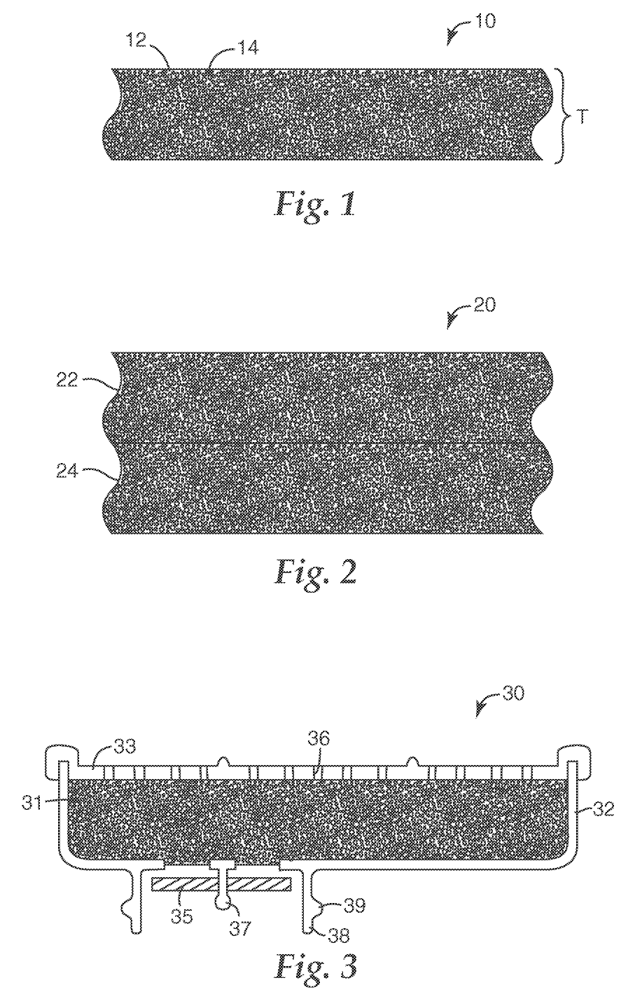

Referring to FIG. 1, a disclosed porous sheet article 10 is shown schematically in cross-section. Article 10 has a thickness T and a length and width of any desired dimension. Article 10 is a nonwoven web containing entangled polymeric fibers 12 and sorbent carbon particles 14 enmeshed in the web. Small connected pores (not identified in FIG. 1) in article 10 permit ambient air or other fluids to pass (e.g., to flow) through the thickness dimension of article 10. Particles 14 absorb solvents and other potentially hazardous substances present in such fluids.

FIG. 2 shows a cross-sectional view of a disclosed multilayer article 20 having two nonwoven layers 22 and 24. Layers 22 and 24 each contain fibers and sorbent particles (not identified in FIG. 2). Layers 22 and 24 may be the same as or different from one another and may be the same as or different from article 10 in FIG. 1. For example, when the sorbent particles in layers 22 and 24 are made from different substances, then different potentially hazardous substances may be removed from fluids passing through article 20. When the sorbent particles in layers 22 and 24 are made from the same substances, then potentially hazardous substances may be removed more effectively or for longer service periods from fluids passing through the thickness dimension article 20 than from a single layer article of equivalent overall composition and thickness. Multilayer articles such as article 20 can if desired contain more than two nonwoven layers, e.g. three or more, four or more, five or more or even 10 or more layers.

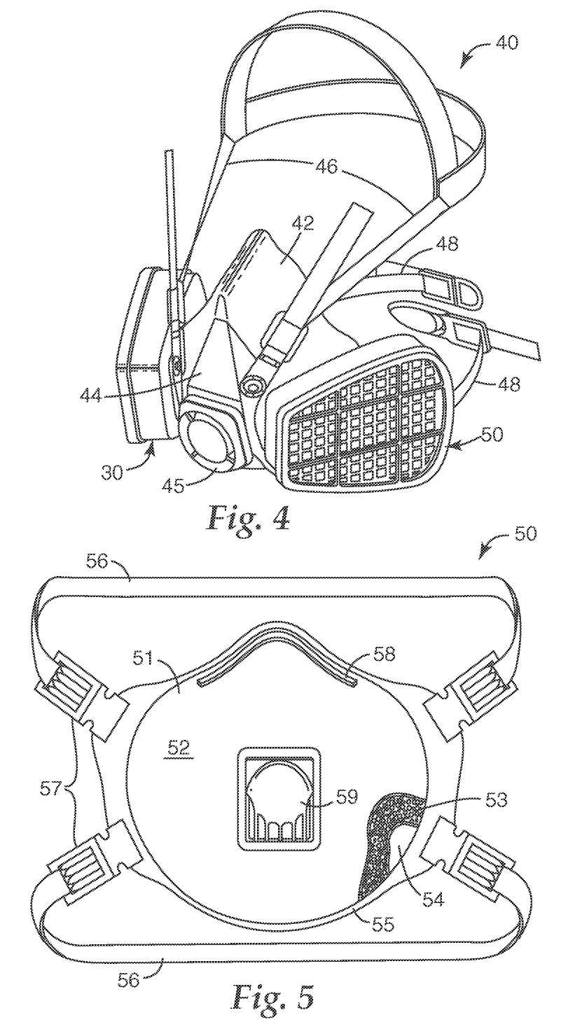

FIG. 3 shows a cross-sectional view of disclosed filter element 30. The interior of element 30 can be filled with a porous sheet article 31 such as those shown in FIG. 1 or FIG. 2. Housing 32 and perforated cover 33 surround sheet article 31. Ambient air enters filter element 30 through openings 36, passes through sheet article 31 (whereupon potentially hazardous substances in such ambient air are absorbed by particles in sheet article 31) and exits element 30 past intake air valve 35 mounted on support 37. Spigot 38 and bayonet flange 39 enable filter element 30 to be replaceably attached to a respiratory device such as disclosed device 40 in FIG. 4. Device 40 is a so-called half mask like that shown in U.S. Pat. No. 5,062,421 (Burns et al.). Device 40 includes soft, compliant face piece 42 that can be insert molded around relatively thin, rigid structural member or insert 44. Insert 44 includes exhalation valve 45 and recessed bayonet-threaded openings (not shown in FIG. 4) for removably attaching filter elements 30 in the cheek regions of device 40. Adjustable headband 46 and neck straps 48 permit device 40 to be securely worn over the nose and mouth of a wearer. Further details regarding the construction of such a device will be familiar to those skilled in the art.

FIG. 5 shows a disclosed respiratory device 50 in partial cross-section. Device 50 is a disposable mask like that shown in U.S. Pat. No. 6,234,171 B1 (Springett et al.). Device 50 has a generally cup-shaped shell or respirator body 51 made from an outer cover web 52, nonwoven web 53 containing sorbent particles such as those shown in FIG. 1 or FIG. 2, and inner cover web 54. Welded edge 55 holds these layers together and provides a face seal region to reduce leakage past the edge of device 50. Device 50 includes adjustable head and neck straps 56 fastened to device 50 by tabs 57, pliable dead-soft metal nose band 58 of a metal such as aluminum and exhalation valve 59. Further details regarding the construction of such a device will be familiar to those skilled in the art.

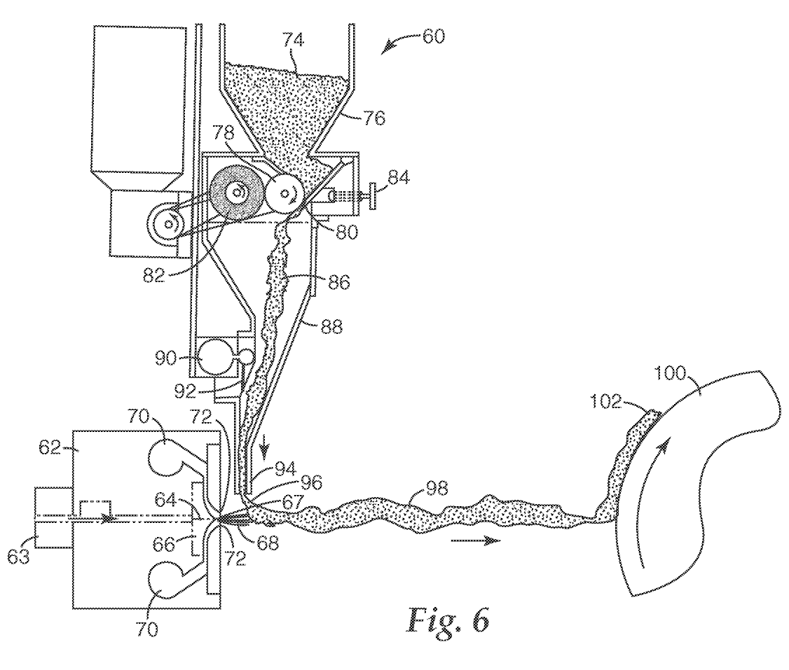

FIG. 6 shows a disclosed apparatus 60 for making nonwoven particle-loaded webs using meltblowing. Molten fiber-forming polymeric material enters nonwoven die 62 via inlet 63, flows through die slot 64 of die cavity 66 (all shown in phantom), and exits die cavity 66 through orifices such as orifice 67 as a series of filaments 68. An attenuating fluid (typically air) conducted through air manifolds 70 attenuates filaments 68 into fibers 98. Meanwhile, sorbent particles 74 pass through hopper 76 past feed roll 78 and doctor blade 80. Motorized brush roll 82 rotates feed roll 78. Threaded adjuster 84 can be moved to improve crossweb uniformity and the rate of particle leakage past feed roll 78. The overall particle flow rate can be adjusted by altering the rotational rate of feed roll 78. The surface of feed roll 78 may be changed to optimize feed performance for different particles. A cascade 86 of sorbent particles 74 falls from feed roll 78 through chute 88. Air or other fluid passes through manifold 90 and cavity 92 and directs the falling particles 74 through channel 94 in a stream 96 amidst filaments 68 and fibers 98. The mixture of particles 74 and fibers 98 lands against porous collector 100 and forms a self-supporting nonwoven particle-loaded meltblown web 102. Further details regarding the manner in which meltblowing would be carried out using such an apparatus will be familiar to those skilled in the art.

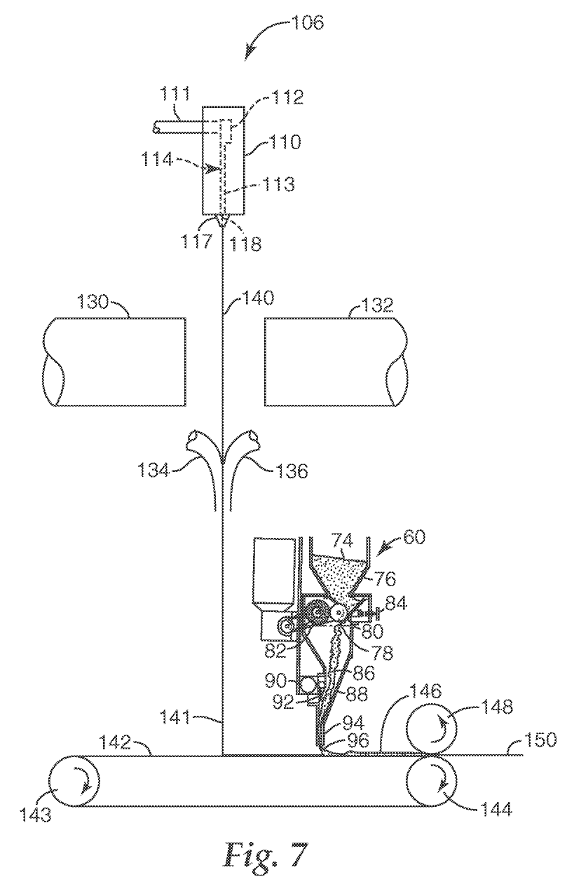

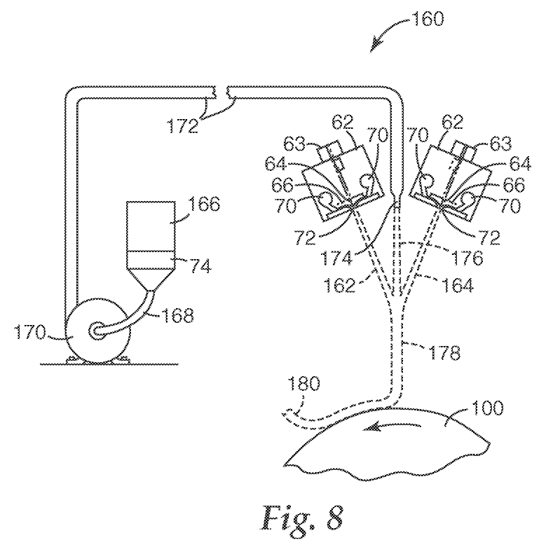

FIG. 7 shows a disclosed apparatus 106 for making nonwoven particle-loaded webs using a spun bond process. Molten fiber-forming polymeric material enters generally vertical nonwoven die 110 via inlet 111, flows downward through manifold 112 and die slot 113 of die cavity 114 (all shown in phantom), and exits die cavity 114 through orifices such as orifice 118 in die tip 117 as a series of downwardly-extending filaments 140. A quenching fluid (typically air) conducted via ducts 130 and 132 solidifies at least the surfaces of the filaments 140. The at least partially solidified filaments 140 are drawn toward collector 142 while being attenuated into fibers 141 by generally opposing streams of attenuating fluid (typically air) supplied under pressure via ducts 134 and 136. Meanwhile, sorbent particles 74 pass through hopper 76 past feed roll 78 and doctor blade 80 in an apparatus like that shown by components 76 through 94 in FIG. 6. Stream 96 of particles 74 is directed through nozzle 94 amidst fibers 141. The mixture of particles 74 and fibers 141 lands against porous collector 142 carried on rollers 143 and 144 and forms a self-supporting nonwoven particle-loaded spun bond web 146. Calendaring roll 148 opposite roll 144 compresses and point-bonds the fibers in web 146 to produce calendared spun bond nonwoven particle-loaded web 150. Further details regarding the manner in which spun bonding would be carried out using such an apparatus will be familiar to those skilled in the art.

FIG. 8 shows a disclosed apparatus 160 for making nonwoven particle-loaded webs using meltblowing. This apparatus employs two generally vertical, obliquely-disposed nonwoven dies 66 that project generally opposing streams of filaments 162, 164 toward collector 100. Meanwhile, sorbent particles 74 pass through hopper 166 and into conduit 168. Air impeller 170 forces air through a second conduit 172 and accordingly draws particles from conduit 168 into the second conduit 172. The particles are ejected through nozzle 174 as particle stream 176 whereupon they mingle with the filament streams 162 and 164 or with the resulting attenuated fibers 178. The mixture of particles 74 and fibers 178 lands against porous collector 100 and forms a self-supporting nonwoven particle-loaded nonwoven web 180. The apparatus shown in FIG. 8 typically will provide a more uniform distribution of sorbent particles than is obtained using the apparatus shown in FIG. 6. Further details regarding the manner in which meltblowing would be carried out using the FIG. 8 apparatus will be familiar to those skilled in the art.

A variety of fiber-forming polymeric materials can be employed, including thermoplastics such as polyurethane elastomeric materials (e.g., those available under the trade designations IROGRAN™ from Huntsman LLC and ESTANE™ from Noveon, Inc.), polybutylene elastomeric materials (e.g., those available under the trade designation CRASTIN™ from E. I. DuPont de Nemours & Co.), polyester elastomeric materials (e.g., those available under the trade designation HYTREL™ from E. I. DuPont de Nemours & Co.), polyether block copolyamide elastomeric materials (e.g., those available under the trade designation PEBAX™ from Atofina Chemicals, Inc.) and elastomeric styrenic block copolymers (e.g., those available under the trade designations KRATON™ from Kraton Polymers and SOLPRENE™ from Dynasol Elastomers). Some polymers may be stretched to much more than 125 percent of their initial relaxed length and many of these will recover to substantially their initial relaxed length upon release of the biasing force and this latter class of materials is generally preferred. Thermoplastic polyurethanes, polybutylenes and styrenic block copolymers are especially preferred. If desired, a portion of the web can represent other fibers that do not have the recited elasticity or crystallization shrinkage, e.g., fibers of conventional polymers such as polyethylene terephthalate; multicomponent fibers (e.g., core-sheath fibers, splittable or side-by-side bicomponent fibers and so-called “islands in the sea” fibers); staple fibers (e.g., of natural or synthetic materials) and the like. Preferably however relatively low amounts of such other fibers are employed so as not to detract unduly from the desired sorbent loading level and finished web properties.

Without intending to be bound by theory, we believe that the elasticity or crystallization shrinkage characteristics of the fiber promote autoconsolidation or densification of the nonwoven web, reduction in the web's pore volume or reduction in the pathways through which gases can pass without encountering an available sorbent particle. Densification may be promoted in some instances by forced cooling of the web using, e.g., a spray of water or other cooling fluid, or by annealing the collected web in an unrestrained or restrained manner. Preferred annealing times and temperatures will depend on various factors including the polymeric fibers employed and the sorbent particle loading level. As a general guide for webs made using polyurethane fibers, annealing times less than about one hour are preferred.

A variety of sorbent particles can be employed. Desirably the sorbent particles will be capable of absorbing or adsorbing gases, aerosols or liquids expected to be present under the intended use conditions. The sorbent particles can be in any usable form including beads, flakes, granules or agglomerates. Preferred sorbent particles include activated carbon; alumina and other metal oxides; sodium bicarbonate; metal particles (e.g., silver particles) that can remove a component from a fluid by adsorption, chemical reaction, or amalgamation; particulate catalytic agents such as hopcalite (which can catalyze the oxidation of carbon monoxide); clay and other minerals treated with acidic solutions such as acetic acid or alkaline solutions such as aqueous sodium hydroxide; ion exchange resins; molecular sieves and other zeolites; silica; biocides; fungicides and virucides. Activated carbon and alumina are particularly preferred sorbent particles. Mixtures of sorbent particles can be employed, e.g., to absorb mixtures of gases, although in practice to deal with mixtures of gases it may be better to fabricate a multilayer sheet article employing separate sorbent particles in the individual layers. The desired sorbent particle size can vary a great deal and usually will be chosen based in part on the intended service conditions. As a general guide, the sorbent particles may vary in size from about 5 to 3000 micrometers average diameter. Preferably the sorbent particles are less than about 1500 micrometers average diameter, more preferably between about 30 and about 800 micrometers average diameter, and most preferably between about 100 and about 300 micrometers average diameter. Mixtures (e.g., bimodal mixtures) of sorbent particles having different size ranges can also be employed, although in practice it may be better to fabricate a multilayer sheet article employing larger sorbent particles in an upstream layer and smaller sorbent particles in a downstream layer. At least 80 weight percent sorbent particles, more preferably at least 84 weight percent and most preferably at least 90 weight percent sorbent particles are enmeshed in the web.

In some embodiments the service life may be affected by whether the collector side of the nonwoven web is oriented upstream or downstream with respect to the expected fluid flow direction. Depending sometimes on the particular sorbent particle employed, improved service lives have been observed using both orientations.

The nonwoven web or filter element has an Adsorption Factor A of at least 1.6×10⁴/mm water. The Adsorption Factor A can be calculated using parameters or measurements similar to those described in Wood, Journal of the American Industrial Hygiene Association, 55(1):11-15 (1994), where:
kᵥ=effective adsorption rate coefficient (min⁻¹) for the capture of C₆H₁₂ vapor by the sorbent according to the equation:

C₆H₁₂ vapor→C₆H₁₂ absorbed on the sorbent.
Wₑ=effective adsorption capacity (g_{C}_{₆}_{H}_{₁₂}/g_{Sorbent}) for a packed sorbent bed or sorbent loaded web exposed to 1000 ppm C₆H₁₂ vapor flowing at 30 L/min (face velocity 4.9 cm/s) and standard temperature and pressure, determined using iterative curve fitting for an adsorption curve plotted from 0 to 50 ppm (5%) C₆H₁₂ breakthrough.
SL=service life (min) for a packed sorbent bed or sorbent loaded web exposed to 1000 ppm C₆H₁₂ vapor flowing at 30 L/min (face velocity 4.9 cm/s) and standard temperature and pressure, based on the time required to reach 10 ppm (1%) C₆H₁₂ breakthrough.
ΔP=pressure drop (mm water) for a packed sorbent bed or sorbent loaded web exposed to air flowing at 85 L/min (face velocity 13.8 cm/s) and standard temperature and pressure.
   The parameter kᵥ is usually not measured directly. Instead, it can be determined by solving for kᵥ using multivariate curve fitting and the equation:

$$\frac{Cx}{Co} = {( 1 + exp [ \frac{kv \times W}{ρβ \times Q} - \frac{kv \times Co \times t}{We \times ρβ \times {10}^{3}} ] )}^{- 1}$$

where
Q=Challenge flow rate (L/min)
Cx=C₆H₁₂ exit concentration (g/L).
Co=C₆H₁₂ inlet concentration (g/L).
W=sorbent weight (g).
t=exposure time.
ρβ=density of a packed sorbent bed or the effective density of a sorbent loaded web where g_{Sorbent} is the weight of sorbent material (excluding the web weight, if present), cm³_{Sorbent} is the overall volume of sorbent, cm³_{Web} is the overall volume of sorbent loaded web, and ρβ has the units g_{Sorbent}/cm³_{Sorbent} for a packed bed or g_{Sorbent}/cm³_{Web} for a sorbent loaded web.
   The Adsorption Factor A can then be determined using the equation:

A=(kᵥ×SL)/ΔP.

The Adsorption Factor may be for example at least 3×10⁴/mm water, at least 4×10⁴/mm water or at least 5×10⁴/mm water. Surprisingly, some embodiments of the invention have Adsorption Factors above those found in a high-quality packed carbon bed, which as shown in Comparative Example 1 below is about 3.16×10⁴/mm water.

A further factor Aᵥₒₗ that relates the Adsorption Factor A to the total product volume can also be calculated. Aᵥₒₗ has the units g_{Sorbent}/cm³_{Web}-mm water, and can be calculated using the equation:

Aᵥₒₗ=A×ρβ

Preferably Aᵥₒₗ is at least about 3×10³ g_{Sorbent}/cm³_{Web}-mm water, more preferably at least about 6×10³ g_{Sorbent}/cm³_{Web}-mm water, and most preferably at least about 9×10³ g_{Sorbent}/cm³_{Web}-mm water.

The invention will now be described with reference to the following non-limiting examples, in which all parts and percentages are by weight unless otherwise indicated.

### Examples 1-20 and Comparative Examples 1-6

Using a meltblowing apparatus with two merging vertical streams of filaments like that shown in FIG. 8, a 210° C. polymer melt temperature, a drilled orifice die and a 28 cm die-to-collector distance, a series of meltblown carbon-loaded nonwoven webs was prepared using various fiber-forming polymeric materials extruded at 143-250 g/hour/cm. The extrusion rate (and as needed, other processing parameters) were adjusted to obtain webs having a 17 to 32 micrometer effective fiber diameter, with most of the webs having a 17 to 23 micrometer effective fiber diameter. The completed webs were evaluated to determine the carbon loading level and the parameters kᵥ, SL, ΔP, ρβ. A and Aᵥₒₗ. The webs were made under varying ambient temperature and humidity conditions and using web-forming equipment located at different sites. Thus a variety of webs having similar ingredients and loading levels were prepared but exhibiting some variation in performance. Comparison data was gathered for a packed carbon bed made from Kuraray Type GG 12×20 activated carbon and for webs made from polypropylene or from polyurethane with a low carbon loading level. Set out below in Table 1 are the Example or Comparative Example number, polymeric material, carbon type, number of meltblowing dies (two for the FIG. 8 apparatus or none for the packed carbon bed shown in Comparative Example 1), carbon loading level and the above-mentioned parameters. The parameters SL and ΔP are expressed as the ratio SL/ΔP. The table entries are sorted according to the A value.

TABLE 1

| Ex. No. | | | No. | | | | | | Aᵥₒₗ, |
|---|---|---|---|---|---|---|---|---|---|
| or | | Carbon, | of | | | SL/ΔP, | | | g_{sorbent}/ |
| Comp. | Polymeric | Sieve | MB | Loading | kᵥ, | min/ | ρβ, | A, | cm³_{Web}- |
| Ex. No. | Material⁽¹⁾ | Size | Dies | Level, % | min⁻¹ | mm H₂O | g/cm³ | /mm water | mm water |
| 1 | PS 440-200 | 12 × 20 | 2 | 91 | 2710 | 22.3 | 0.22 | 60433 | 13295 |
| 2 | PS 440-200 | 12 × 40 | 2 | 91 | 2867 | 20.3 | 0.24 | 58200 | 13968 |
| 3 | PS 440-200 | 12 × 20 | 2 | 91 | 2309 | 23.3 | 0.22 | 53800 | 11836 |
| 4 | PS 440-200 | 12 × 20 | 2 | 84 | 2359 | 22.0 | 0.21 | 51898 | 10899 |
| 5 | PS 440-200 | 40 × 140 | 2 | 91 | 6584 | 6.6 | 0.20 | 43454 | 8691 |
| 6 | PS 440-200 | 12 × 20 | 2 | 91 | 2077 | 20.5 | 0.22 | 42579 | 9367 |
| 7 | PS 440-200 | 40 × 140 | 2 | 91 | 5790 | 7.0 | 0.20 | 40530 | 8106 |
| 8 | PS 164-200 | 40 × 140 | 2 | 91 | 6837 | 5.8 | 0.19 | 39655 | 7534 |
| 9 | PS 440-200 | 40 × 140 | 2 | 86 | 7849 | 5.0 | 0.18 | 39245 | 7064 |
| 10 | PS 164-200 + | 40 × 140 | 2 | 91 | 6812 | 5.7 | 0.20 | 38828 | 7766 |
| | PS 440-200 | | | | | | | | |
| 11 | PS 440-200 | 12 × 20 | 2 | 91 | 1991 | 19.2 | 0.23 | 38227 | 8792 |
| 12 | PS 440-200 | 75/25 | 2 | 91 | 3306 | 10.8 | 0.21 | 35705 | 7498 |
| | | blend | | | | | | | |
| | | 12 × 20/ | | | | | | | |
| | | 40 × 140 | | | | | | | |
| 13 | PS 440-200 | 40 × 140 | 2 | 88 | 7017 | 4.8 | 0.18 | 33682 | 6063 |
| 14 | PS 440-200 | 60/40 | 2 | 92 | 3355 | 10.0 | 0.22 | 33550 | 7381 |
| | | blend | | | | | | | |
| | | 12 × 20/ | | | | | | | |
| | | 40 × 140 | | | | | | | |
| 15 | PS 440-200 | 12 × 40 | 2 | 91 | 2738 | 11.3 | 0.22 | 30939 | 6807 |
| Comp. 1 | None (packed | 12 × 20 | 0 | 100 | 7220 | 4.1 | 0.43 | 29602 | 12729 |
| | bed) | | | | | | | | |
| 16 | PS 440-200 | 12 × 20 | 2 | 91 | 1908 | 14.3 | 0.20 | 27284 | 5457 |
| 17 | PS 440-200 | 12 × 20 | 2 | 91 | 1843 | 14.7 | 0.20 | 27092 | 5418 |
| 18 | PS 440-200 | 12 × 20 | 2 | 90 | 1895 | 11.5 | 0.20 | 21793 | 4359 |
| 19 | PS 440-200 | 12 × 20 | 2 | 90 | 1649 | 13.1 | 0.18 | 21602 | 3888 |
| 20 | PS 440-200 | 12 × 20 | 2 | 88 | 1608 | 10.5 | 0.17 | 16884 | 2870 |
| Comp. 2 | F3960 | 12 × 20 | 2 | 91 | 1352 | 11.4 | 0.15 | 15413 | 2312 |
| Comp. 3 | F3960 | 40 × 140 | 2 | 89 | 3642 | 4.2 | 0.14 | 15296 | 2141 |
| Comp. 4 | F3960 | 12 × 20 | 2 | 91 | 1442 | 10.1 | 0.16 | 14564 | 2330 |
| Comp. 5 | PS 440-200 | 40 × 140 | 2 | 78 | 4815 | 2.1 | 0.13 | 10112 | 1315 |
| Comp. 6 | F3960 | 12 × 20 | 2 | 89 | 927 | 8.4 | 0.11 | 7787 | 857 |

⁽¹⁾PS 440-200 is a thermoplastic polyurethane (commercially available from Huntsman LLC). PS 164-200 is a thermoplastic polyurethane (commercially available from Huntsman LLC). F3960 is FINA ™ 3960 polypropylene homopolymer (commercially available from Atofina Chemicals, Inc.).

The data in Table 1 show that very high Adsorption Factor A values could be obtained, in many cases exceeding the Adsorption Factor A for a packed carbon bed. Webs made from polypropylene (Comparative Example Nos. 2-4 and 6) and webs made using an elastomeric fiber but with less than about 80 wt. % carbon (Comparative Example No. 5) had lower Adsorption Factor A values. For example, webs made using PS 440-200 polyurethane loaded with 91 wt. % 12×20 carbon had Adsorption Factor A values between 27,092 and 60,433/mm water, whereas the best performing web made using FINA 3960 polypropylene and 91 wt. % 12×20 carbon had an Adsorption Factor A of only 15,413/mm water (compare Example Nos. 1 and 17 to Comparative Example No. 2). This performance advantage was maintained even when compared to polyurethane webs made using a lower carbon level (compare e.g., Example No. 4 to Comparative Example No. 2) so long as the carbon level did not fall below about 80 wt. % (see, e.g., Comparative Example No. 5).

### Examples 21-41 and Comparative Examples 7-30

Using a meltblowing apparatus with a single horizontal stream of filaments like that shown in FIG. 6, a 210° C. polymer melt temperature, a drilled orifice die and a 30.5 cm die-to-collector distance, a series of meltblown carbon-loaded nonwoven webs was prepared using various fiber-forming polymeric materials extruded at 143-250 g/hour/cm. The extrusion rate (and as needed, other processing parameters) were adjusted to obtain webs having a 14 to 24 micrometer effective fiber diameter, with most of the webs having a 17 to 23 micrometer effective fiber diameter. The completed webs were evaluated to determine the carbon loading level and the parameters kᵥ, SL, ΔP, ρβ, A and Aᵥₒₗ. Set out below in Table 2 along with data from Table 1 for Comparative Example 1 are the Example or Comparative Example number, polymeric material, carbon type, number of meltblowing dies (one for the FIG. 6 apparatus or none for the packed carbon bed shown in Comparative Example 1), carbon loading level and the above-mentioned parameters. The parameters SL and ΔP are expressed as the ratio SL/ΔP. The table entries are sorted according to the A value.

TABLE 2

| | | | No. | | | | | | Aᵥₒₗ, |
|---|---|---|---|---|---|---|---|---|---|
| Ex. No. | | Carbon, | of | | | SL/ΔP, | | | g_{sorbent}/ |
| or Comp. | Polymeric | Sieve | MB | Loading | kᵥ, | min/ | ρβ, | A, | cm³_{Web}- |
| Ex. No. | Material⁽²⁾ | Size | Dies | Level, % | min⁻¹ | mm H₂O | g/cm³ | /mm water | mm water |
| 21 | PS 440-200 | 12 × 20 | 1 | 91 | 1946 | 17 | 0.21 | 33082 | 6947 |
| 22 | PS 440-200 | 12 × 40 | 1 | 91 | 3027 | 10.5 | 0.21 | 31784 | 6675 |
| Comp. 1 | None (packed | 12 × 20 | 0 | 100 | 7220 | 4.1 | 0.43 | 29602 | 12729 |
| | bed) | | | | | | | | |
| 23 | G3548L | 12 × 20 | 1 | 90 | 1787 | 15.8 | 0.19 | 28235 | 5365 |
| 24 | PS 440-200 | 40 × 140 | 1 | 91 | 6569 | 4 | 0.22 | 26276 | 5781 |
| 25 | PS 440-200 | 16 × 35 | 1 | 91 | 3824 | 6.8 | 0.22 | 26003 | 5721 |
| 26 | PS 440-200 | 12 × 20 | 1 | 91 | 1678 | 14.7 | 0.18 | 24667 | 4440 |
| 27 | 50% F3868 + | 12 × 20 | 1 | 90 | 1726 | 13.5 | 0.20 | 23301 | 4660 |
| | 50% PB 0400 | | | | | | | | |
| 28 | 50% F3868 + | 12 × 20 | 1 | 90 | 1757 | 13.2 | 0.20 | 23192 | 4638 |
| | 50% PB 0400 | | | | | | | | |
| 29 | PS 440-200 | 40 × 140 | 1 | 91 | 7909 | 2.8 | 0.21 | 22145 | 4650 |
| 30 | PS 440-200 | 12 × 20 | 1 | 90 | 1875 | 11.8 | 0.18 | 22125 | 3983 |
| 31 | PS 440-200 | 12 × 20 | 1 | 90 | 1858 | 11.9 | 0.20 | 22110 | 4422 |
| 32 | G3548L | 40 × 140 | 1 | 88 | 7880 | 2.8 | 0.19 | 22064 | 4192 |
| 33 | G3548L | 12 × 20 | 1 | 88 | 1664 | 12.9 | 0.18 | 21466 | 3864 |
| 34 | G3548L | 12 × 20 | 1 | 90 | 1739 | 12.2 | 0.19 | 21216 | 4031 |
| 35 | G3548L | 40 × 140 | 1 | 87 | 8050 | 2.5 | 0.20 | 20125 | 4025 |
| 36 | PS 440-200 | 40 × 140 | 1 | 81 | 8490 | 2.3 | 0.20 | 19527 | 3905 |
| 37 | 100% PB 0400 | 12 × 20 | 1 | 90 | 1868 | 10.1 | 0.20 | 18864 | 3716 |
| 38 | 20% 3868 + | 12 × 20 | 1 | 89 | 1922 | 9.7 | 0.20 | 18643 | 3729 |
| | 80% PB 0400 | | | | | | | | |
| 39 | PS 440-200 | 40 × 140 | 1 | 92 | 5413 | 3.3 | 0.17 | 17863 | 3037 |
| 40 | 100% PB 0400 | 12 × 20 | 1 | 90 | 1802 | 9.4 | 0.20 | 16936 | 3336 |
| 41 | 100% PB 0400 | 12 × 20 | 1 | 90 | 1759 | 9.3 | 0.20 | 16356 | 3222 |
| Comp. 7 | 100% PB 0400 | 12 × 20 | 1 | 90 | 1861 | 8.2 | 0.20 | 15262 | 3007 |
| Comp. 8 | PS 440-200 | 40 × 140 | 1 | 90 | 5422 | 2.8 | 0.19 | 15182 | 2885 |
| Comp. 9 | 20% 3868 + | 12 × 20 | 1 | 89 | 1833 | 8.1 | 0.20 | 14847 | 2969 |
| | 80% PB 0400 | | | | | | | | |
| Comp. 10 | F3960 | 12 × 20 | 1 | 90 | 1311 | 11.3 | 0.15 | 14814 | 2222 |
| Comp. 11 | F3960/E-1200 | 40 × 140 | 1 | 90 | 3834 | 3.8 | 0.16 | 14569 | 2331 |
| Comp. 12 | PS 440-200 | 40 × 140 | 1 | 91 | 5567 | 2.6 | 0.18 | 14474 | 2605 |
| Comp. 13 | F3960 | 40 × 140 | 1 | 91 | 4478 | 3.2 | 0.17 | 14330 | 2436 |
| Comp. 14 | F3960 | 40 × 140 | 1 | 89 | 3588 | 3.8 | 0.14 | 13634 | 1909 |
| Comp. 15 | G-1657 | 12 × 20 | 1 | 88 | 2422 | 5.6 | 0.22 | 13563 | 2984 |
| Comp. 16 | PS 440-200 | 40 × 140 | 1 | 66 | 8844 | 1.5 | 0.15 | 13266 | 1990 |
| Comp. 17 | PS 440-200 | 12 × 20 | 1 | 81 | 1563 | 7.7 | 0.16 | 12035 | 1926 |
| Comp. 18 | PS 440-200 | 12 × 20 | 1 | 87 | 1776 | 6.5 | 0.18 | 11541 | 2077 |
| Comp. 19 | F3960/E-1200 | 12 × 20 | 1 | 90 | 1389 | 8.3 | 0.16 | 11525 | 1844 |
| Comp. 20 | G3548L | 12 × 20 | 1 | 82 | 1748 | 6.2 | 0.16 | 10836 | 1734 |
| Comp. 21 | F3960 | 12 × 20 | 1 | 90 | 1348 | 8 | 0.15 | 10784 | 1618 |
| Comp. 22 | F3960 | 12 × 20 | 1 | 91 | 1440 | 7.2 | 0.15 | 10368 | 1555 |
| Comp. 23 | D2503 | 12 × 20 | 1 | 90 | 1942 | 5.3 | 0.19 | 10290 | 1955 |
| Comp. 24 | F3960 | 40 × 140 | 1 | 89 | 3271 | 2.7 | 0.14 | 8832 | 1236 |
| Comp. 25 | PS 440-200 | 12 × 20 | 1 | 84 | 1662 | 5.2 | 0.16 | 8640 | 1382 |
| Comp. 26 | F3960 | 12 × 20 | 1 | 91 | 1216 | 6.3 | 0.14 | 7659 | 1072 |
| Comp. 27 | PS 440-200 | 40 × 140 | 1 | 49 | 6035 | 1.2 | 0.11 | 7242 | 797 |
| Comp. 28 | PS 440-200 | 40 × 140 | 1 | 50 | 6830 | 0.8 | 0.12 | 5464 | 656 |
| Comp. 29 | PS 440-200 | 12 × 20 | 1 | 68 | 1333 | 3.3 | 0.14 | 4399 | 616 |
| Comp. 30 | PS 440-200 | 12 × 20 | 1 | 50 | 1216 | 1.2 | 0.13 | 1459 | 190 |

⁽²⁾PS 440-200 is a thermoplastic polyurethane (commercially available from Huntsman LLC). G3548L is HYTREL ™ G3548L thermoplastic poly butylene/poly(alkylene ether) phthalate elastomer (commercially available from DuPont Plastics). F3848 is FINA 3868 polypropylene homopolymer (commercially available from Atofina Chemicals, Inc.). PB 0400 is POLYBUTENE-1 ™ Grade PB 0400 thermoplastic polybutylene elastomer (commercially available from Basell Polyolefins). G-1657 is KRATON ™ G-1657 styrenic di-/triblock copolymer (commercially available from Kraton Polymers). F3960 is FINA 3960 polypropylene homopolymer (commercially available from Atofina Chemicals, Inc.). E-1200 is EASTOFLEX ™ E-1200 amorphous propylene-ethylene copolymer (commercially available from Eastman Chemicals). D2503 is DOWLEX ™ 2503 linear low density low molecular weight polyethylene resin (commercially available from Dow Plastics).

The data in Table 2 show that very high Adsorption Factor A values could be obtained. However, the values typically were lower than those shown in Table 1. In some instances webs made using materials and amounts like those employed in Table 1 and containing more than 80 wt. % carbon particles did not exhibit an Adsorption Factor A of at least 1.6×10⁴/mm water (compare e.g., Example 5 and Comparative Example No. 12). This was believed to be at least partly due to a visibly less uniform distribution of carbon particles within the Table 2 webs, and may also have been at least partly due to the use of a single layer web rather than a two layer web.

### Examples 42-43 and Comparative Examples 31-32

Using a meltblowing apparatus with a single horizontal stream of filaments like that used in Examples 21-41 and a post-collection vacuum forming step to consolidate the resulting webs, a series of meltblown carbon-loaded nonwoven webs was prepared using various fiber-forming polymeric materials and evaluated to determine the carbon loading level and the parameters kV, SL, ΔP, ρβ, A and Aᵥₒₗ. Set out below in Table 3 along with data from Table 1 for Comparative Example 1 are the Example or Comparative Example number, polymeric material, carbon type, number of meltblowing dies (one for the FIG. 6 apparatus or none for the packed carbon bed shown in Comparative Example 1), carbon loading level and the above-mentioned parameters. The parameters SL and ΔP are expressed as the ratio SL/ΔP. The table entries are sorted according to the A value.

TABLE 3

| | | | No. | | | | | | Aᵥₒₗ, |
|---|---|---|---|---|---|---|---|---|---|
| Ex. No. or | | Carbon, | of | | | SL/ΔP, | | | g_{sorbent}/ |
| Comp. Ex. | Polymeric | Sieve | MB | Loading | kᵥ, | min/ | ρβ, | A, | cm³_{Web}- |
| No. | Material⁽³⁾ | Size | Dies | Level, % | min⁻¹ | mm H₂O | g/cm³ | /mm water | mm water |
| 42 | PS 440-200 | 12 × 20 | 1 | 91 | 2357 | 16.5 | 0.23 | 38895 | 8946 |
| Comp. 1 | None | 12 × 20 | 0 | 100 | 7220 | 4.1 | 0.43 | 29602 | 12729 |
| | (packed | | | | | | | | |
| | bed) | | | | | | | | |
| Comp. 31 | F3960 | 12 × 20 | 1 | 89 | 1389 | 15.3 | 0.15 | 21252 | 3188 |
| 43 | PS 440-200 | 12 × 20 | 1 | 90 | 1898 | 10.9 | 0.19 | 20687 | 3931 |
| Comp. 32 | F3960 | 12 × 20 | 1 | 91 | 1650 | 12.3 | 0.17 | 20297 | 3532 |

⁽³⁾PS 440-200 is a thermoplastic polyurethane (commercially available from Huntsman LLC). F3960 is FINA 3960 polypropylene homopolymer (commercially available from Atofina Chemicals, Inc.).

The results in Table 3 show that using a vacuum post-forming technique to consolidate the web may provide an improvement in the Adsorption Factor A (compare e.g., Example 42 to Example 21 and Comparison Examples 31 and 32 to Comparison Example 10). This improvement was not always observed (compare e.g., Example 43 to Examples 30 and 31).

### Example 44

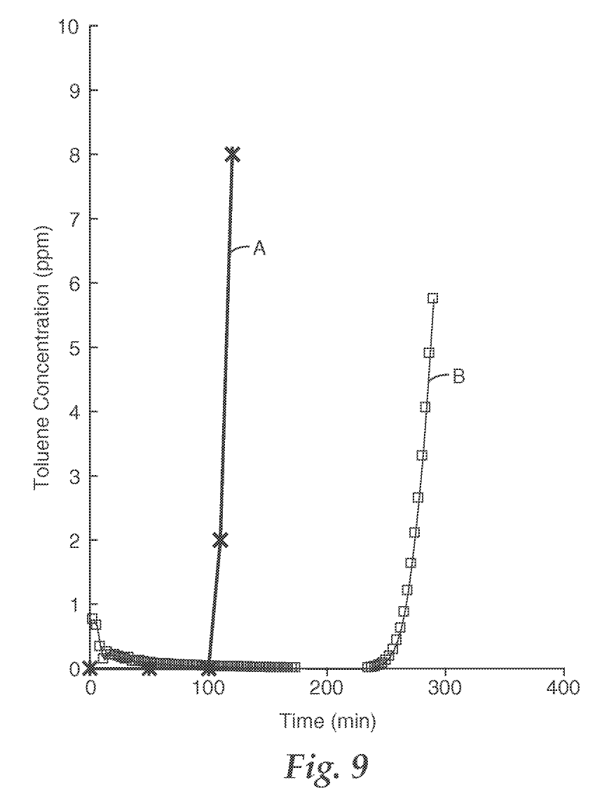

Using the general method of Example 21, a single layer web was made using PS 440-200 thermoplastic polyurethane and 40×140 carbon granules. The completed web contained 0.202 g/cm² carbon (91 wt. % carbon) and had a 15 micrometer effective fiber diameter. Using the method of U.S. Pat. No. 3,971,373 (Braun) Example 19, an 81 cm² sample of the Example 46 web containing 16.3 g total carbon was exposed to <35% relative humidity air flowing at 14 L/min and containing 250 ppm toluene vapor. FIG. 9 shows a plot of the downstream toluene concentration for the Example 44 web (Curve B) and a plot of the Braun Example 19 downstream toluene concentration (Curve A). The Braun Example 19 web contained polypropylene fibers and 17.4 g total carbon (89 wt. % carbon). As shown in FIG. 9 it exhibited substantially less adsorption capacity than the Example 44 web, even though the Example 44 web contained less carbon.

### Example 45

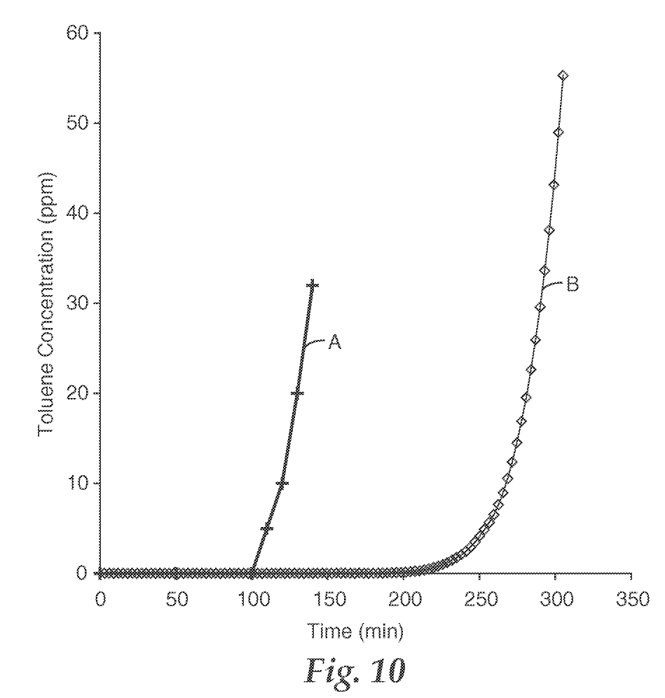

Using the general method of Example 21, a two layer web was made using PS 440-200 thermoplastic polyurethane, 12×20 carbon granules in the first layer and 40×140 carbon granules in the second layer. The first layer contained 0.154 g/cm² carbon (91 wt. % carbon) and had a 26 micrometer effective fiber diameter. The second layer contained 0.051 g/cm² carbon (91 wt. % carbon) and had a 15 micrometer effective fiber diameter. Using the method of U.S. Pat. No. 3,971,373 (Braun) Example 20, an 81 cm² sample of the Example 45 web containing 16.6 g total carbon was exposed to <35% relative humidity air flowing at 14 L/min and containing 350 ppm toluene vapor. FIG. 10 shows a plot of the downstream toluene concentration for the Example 45 web (Curve B) and a plot of the Braun Example 20 downstream toluene concentration (Curve A). The Braun Example 20 web contained polypropylene fibers and 18.9 g total carbon (85 wt. % carbon). As shown in FIG. 10 it exhibited substantially less adsorption capacity than the Example 45 web, even though the Example 45 web contained less carbon.

Various modifications and alterations of this invention will be apparent to those skilled in the art without departing from this invention. This invention should not be restricted to that which has been set forth herein only for illustrative purposes.

We claim:

1. A porous sheet article comprising a self-supporting nonwoven web of polymeric fibers and at least 80 weight percent sorbent particles enmeshed in the web, the fibers having sufficiently greater elasticity or sufficiently greater crystallization shrinkage than similar caliper polypropylene fibers and the sorbent particles being sufficiently evenly distributed in the web so that the web has an Adsorption Factor A of at least 1.6×10⁴/mm water.

2. An article according to claim 1 comprising a plurality of nonwoven web layers.

3. An article according to claim 1 wherein the fibers comprise a thermoplastic polyurethane elastomer.

4. An article according to claim 1 wherein the fibers comprise a thermoplastic polybutylene elastomer.

5. An article according to claim 1 wherein the fibers comprise a thermoplastic polyester elastomer.

6. An article according to claim 1 wherein the fibers comprise a thermoplastic styrenic block copolymer.

7. An article according to claim 1 wherein the sorbent particles comprise activated carbon or alumina.

8. An article according to claim 1 wherein at least 84 weight percent sorbent particles are enmeshed in the web.

9. An article according to claim 1 wherein at least 90 weight percent sorbent particles are enmeshed in the web.

10. An article according to claim 1 having an Adsorption Factor A of at least 3×10⁴/mm water.

11. An article according to claim 1 having an Adsorption Factor A of at least 4×10⁴/mm water.

12. An article according to claim 1 having an Adsorption Factor A of at least 5×10⁴/mm water.
